(19) United States
(12) Patent Application Publication
(10) Pub. No.: US 2009/0203780 A1
(43) Pub. Date: Aug. 13, 2009

(54) Use of a Polyunsaturated Fatty Acid Compound

(76) Inventors: Luisa Gambelli, Wormerveer (NL); Ulrike Schmid, Wormerveer (NL); Alexandra Wilhelmina Carla Einerhand, Wormerveer (NL)

Correspondence Address: MORGAN LEWIS & BOCKIUS LLP, 1111 PENNSYLVANIA AVENUE NW, WASHINGTON, DC, 20004, US

(21) Appl. No.: 12/306,292

(22) PCT Filed: Jun. 26, 2007
(86) PCT No.: PCT/EP07/05644

§ 371 Date: Apr. 15, 2009

(30) Foreign Application Priority Data
Jun. 27, 2006 (EP) 06253339.3

(51) Int. Cl. A61K 31/232; A61K 31/202; A61P 3/04

(52) U.S. Cl. 514/549; 514/560

(57) ABSTRACT

A polyunsaturated fatty acid having 20 carbon atoms and four double bonds, or a derivative thereof, in an orally administrable form, can be used to treat or prevent obesity and/or for weight management. The polyunsaturated fatty acid may, for example, be used in the form of a food supplement, a pharmaceutical composition or a food composition.

This invention relates to compositions for oral administration that may be used for treating or preventing obesity and/or for weight management. The compositions contain a polyunsaturated fatty acid or a derivative thereof.

Obesity is becoming increasingly prevalent in individuals in developed societies. Generally, an overweight condition and obesity result from an imbalance in energy intake and utilisation. The cause of energy imbalance for each individual may be due to a combination of several factors and stems from a myriad of both environmental and genetic determinants. Obesity may be a contributing factor in the increased incidence of various diseases including coronary artery disease, hypertension, stroke, diabetes and certain cancers.

Weight reduction is often recommended as the first course of action for patients suffering from these obesity-related diseases. In an attempt to control total body weight, an individual may undertake weight management, the objectives of which may differ depending on the needs of the individual. For example, whereas obese or overweight individuals may wish to lose body weight, other individuals may wish to maintain a body weight at a substantially constant level. Even once a person has lost body weight, weight management is often required to prevent that person regaining the body weight previously lost. The most effective weight loss programmes typically include a reduced calorie diet and/or increased energy expenditure. Over time, many people undertaking weight management experience increased hunger levels and/or cravings for high sugar foods. This can lead individuals to stray from their weight management regime. There is, therefore, a need to develop new and effective ways to support weight management and to help individuals continue with their weight management regime. It is an object of the present invention to develop a new means for providing this support.

Arachidonic acid, which is also known as 5,8,11,14-eicosatetraenoic acid, is a long chain polyunsaturated fatty acid (PUFA) of the omega-6 class, having 20 carbon atoms and four double bonds (i.e., C20:4 PUFA) at positions 5, 8, 11 and 14. Arachidonic acid is one of the most abundant C20 PUFAs in the human body. It is particularly prevalent in organs, muscles and blood tissues, serving a major role as a structural lipid associated predominately with phospholipids in the blood, liver, muscle and other major organs.

Various physiological functions of arachidonic acid have been reported, e.g., protection of gastric mucosa (Hollander et al, (1982), J. Lab. Clin. Med., 100:296-308 and Doyle et al, (1989), Prostaglandins, 38:581-597); treatment of skin psoriasis (Hebborn et al, (1988), Arch. Dermatol., 124:387-391); reduction of fatty liver (Goheen et al, (1980), Lipids 15:328-336) and; the killing of tumor cells (Canuto et al, Cancer Res., 51:4603-4608).

In addition to the above-mentioned biological activities, the nutritional aspect of arachidonic acid has also been highlighted along with other PUFAs. Arachidonic acid has been extensively investigated, particularly as a substance essential for the growth of infants, see, for example, EP-A-1188383.

According to EP-A-1542670, arachidonic acid and/or compounds containing arachidonic acid as a constituent fatty acid may be used to obtain compounds that prevent decline of, or improve or enhance normal responses of, cognitive abilities of a healthy person.

U.S. Pat. No. 6,429,190, US 2002/0119948 and US 2002/0119915 describe compositions for extending satiety comprising a calcium source, protein and a C12 to C18 fatty acid. Oleic acid is the fatty acid described in the documents.

WO 02/088159 discloses pharmaceutically active uridine esters, and combinations comprising their constituent acid and uridine compound, and their use in a wide variety of medical applications. There is no disclosure of the use of free fatty acids alone nor is any example given of the treatment of obesity.

EP-A-1504778, published on 9 Feb. 2005, describes an implantable pump for the treatment of obesity. The pump may comprise a fatty acid.

CN-A-1377673 relates to the use of a pine nut oil for treating cardiac and cerebral vascular diseases and adiposity caused by hyperlipemia as well as diabetes caused by hyperglycemia.

Lawton et al, (2000), British Journal of Nutrition, 83, 473-482 discloses that the degree of saturation of fatty acids influences post-ingestive satiety.

WO 02/01969 relates to nutritional and pharmaceutical formulations comprising vitamin K and a source of at least one essential fatty acid.

WO 02/098413 discloses a pharmaceutical composition comprising a solid pharmaceutically active compound, such as a lipase inhibitor, and a fatty acid or a fatty acid salt or a mixture thereof.

U.S. Pat. No. 6,071,544 describes a dietary composition for cats comprising a long chain essential fatty acid and protein.

US 2002/0081315 relates to the use of a composition comprising at least one chromium complex and a conjugated fatty acid or conjugated fatty alcohol.

WO 2004/082402 discloses a combined preparation, pharmaceutical or nutritional composition, which comprises at least one cis-polyunsaturated fatty acid and at least one amino acid.

EP-A-1442741 relates to a dietary composition comprising a zinc salt together with cyclo-Hispro and/or arachidonic acid.

Lal, Arch Int Pharmacodyn Ther., (1984), 272(1):140-9 studies the effect of subcutaneously injected fatty acids and prostaglandin synthetase inhibitors on food intake in newborn and young rats.

Our copending application, EP-A-1685834, describes the use of pinolenic acid and its derivatives for weight management.

There remains a need for effective and active compounds for treating or preventing obesity and/or for weight management.

It has now surprisingly been found that C20:4 PUFAs and derivatives thereof can be used to treat or prevent obesity and/or manage weight and are surprisingly highly-effective. None of the prior art documents cited above indicate that a C20:4 PUFA or a derivative thereof could be used to treat or prevent obesity and/or for weight management by oral administration.

According to the invention, there is provided the use of a polyunsaturated fatty acid having 20 carbon atoms and four double bonds, or a derivative thereof, in the manufacture of an orally administrable composition for treating or preventing obesity and/or for weight management.

In another aspect, the invention provides a method of treating or preventing obesity comprising administering an effective amount of the composition of the present invention to a mammal.

In yet another aspect, the invention provides a method of weight management comprising administering an effective amount of the composition of the present invention to a mammal.

A further aspect of the invention is a method of stimulating the production of cholecystokinin (CCK) comprising administering an effective amount of the composition of the present invention to a mammal.

Another aspect of the invention is a composition for oral administration comprising: from 0.5 to 20 wt %, more preferably from 1 to 15 wt % of arachidonic acid or a derivative thereof; and from 0.5 to 40 wt %, more preferably from 1 to 30 wt % of pinolenic acid or a derivative thereof.

The present invention provides the use of a C20:4 PUFA or a derivative thereof in the manufacture of an orally administrable composition for treating or preventing obesity and/or for weight management. The composition preferably works by reducing the feeling of hunger and/or increasing satiety. The invention also provides the use of a C20:4 PUFA or a derivative thereof for reducing the feeling of hunger and/or increasing satiety. Thus, compositions of the invention are suitable for treating or preventing obesity and/or for weight management and comprise a C20:4 PUFA or a derivative thereof. The preferred C20:4 PUFA is arachidonic acid. Arachidonic acid is available commercially, for example as a microbial oil.

The C20:4 PUFA used in the present invention may be in the form of a free fatty acid, a derivative of the C20:4 PUFA or mixtures thereof, including mixtures of different, derivatives. Derivatives are non-toxic and edible and preferably include, for example, salts and esters. Suitable salts include salts with food grade cations such as sodium salts. Suitable esters include alkyl esters having from one to six carbon atoms. Preferred esters are mono-, di- and tri-glycerides, and mixtures thereof, with triglycerides being particularly preferred.

The invention also contemplates a C20:4 PUFA or a derivative thereof for treating or preventing obesity and/or for weight management. The C20:4 PUFA or derivative thereof may be used alone or may be one component of a composition which comprises other edible and/or pharmaceutically acceptable components.

It has surprisingly been found that C20:4 PUFAs can be used to stimulate the release of cholecystokinin (CCK). CCK is a peptide released following the consumption of food. When food is consumed, CCK releasing protein (CCKRP) is released in the small intestine. CCKRP stimulates CCK release from intestinal cells. The release of CCK generates the behavioural symptoms associated with satiety. Additionally, increased CCK levels can increase IgA levels in the gut that can increase mucosal immunity. Increased levels of IgA in the intestinal tract may offer increased protection against invading microorganisms. Therefore, the invention also contemplates a method of increasing immunity (e.g., mucosal immunity) comprising the administration of a C20:4 PUFA or a derivative thereof for increasing immunity (e.g., mucosal immunity) and the use of a C20:4 PUFA or a derivative thereof in the manufacture of a composition for increasing immunity (e.g., mucosal immunity). Increasing immunity may be used in the treatment and/or prevention of infections, such as bacterial, or viral infections.

In broad terms, the invention relates to the use of straight chain carboxylic acids (and their derivatives such as salts and esters) having 20 carbon atoms and four double bonds for weight management and/or for treating or preventing obesity. Weight management may comprise reducing the feeling of hunger and/or increasing satiety, resulting in a reduction of food intake.

Typically, the C20:4 PUFA or derivative thereof that is used in the compositions of the present invention is arachidonic acid or a derivative thereof. Although arachidonic acid is particularly preferred, other C20:4 PUFAs may also be used. For example, geometric isomers of arachidonic acid having one or more trans double bonds (the double bonds in arachidonic acid are all cis) and/or a C20:4 PUFA with one or more of the four double bonds at a different position in the alkyl chain compared to arachidonic acid, and their derivatives, may be used in the invention.

The C20:4 PUFA or derivative for use in the compositions of the present invention is preferably obtained from one or more organisms or microorganisms capable of producing arachidonic acid. Preferably, the C20:4 PUFA or derivative thereof is obtained from algae.

The compositions of the invention are orally administrable (i.e., they are adapted for oral administration) and may be in any suitable form such as a food supplement, a pharmaceutical composition or a food composition.

The term composition means that the C20:4 PUFA or derivative thereof may be present with one or more other components. The one or more other components may be present in admixture with the C20:4 PUFA or derivative or they may form part of the packaging of the product (e.g., the capsule in which the C20:4 PUFA or derivative is encapsulated).

Compositions of the invention may comprise from 0.1 to 100% by weight arachidonic acid or derivative thereof, such as from 1 to 80%, more preferably from 5 to 70%, such as from 10 to 60% by weight based on the weight of the composition. The total weight of arachidonic acid or derivative in the compositions of the invention is preferably from 1 mg to 1000 mg, such as from 10 mg to 750 mg, e.g., from 100 mg to 400 mg.

Compositions of the invention may comprise one or more fatty acids or fatty acid derivatives in addition to the C20:4 PUFA or derivative. The compositions of the present invention preferably comprise from 0.3 to 100 wt % (more preferably from 15 to 80 wt %, most preferably from 30 to 60 wt %, such as from 40 to 55 wt %) of arachidonic acid; from 0.5 to 20 wt % (more preferably from 1 to 15 wt %, such as from 1 to 1.0 wt % or 2 to 8 wt %) of linoleic acid; and optionally from 0.5 to 40 wt % (more preferably from 1 to 35 wt %, such as from 1 to 25 wt % or 3 to 17 wt %) oleic acid. The compositions may further comprise from 0.5 to 25 wt % (more preferably from 1 to 20 wt %, such as from 1 to 15 wt % or 3 to 10 wt %) palmitic acid. Another optional component is from 0.5 to 25 wt %, such as from 1 to 15 wt % or 2 to 10 wt % stearic acid. Additionally or alternatively, the composition may comprise from 0.5 to 10 wt % of gamma linolenic acid and/or from 0.5 to 15 wt % of homogamma linolenic acid. The optional further fatty acids may be present in the compositions of the present inventions either as free acids or glycerides (e.g., mono-, di- or tri-glycerides). Weight percentages in this regard are calculated as free fatty acids based on the total fatty acid content of the composition and the balance to 100% is provided by the remaining fatty acids.

In another preferred embodiment, the C20:4 PUFA or derivative is used together with pinolenic acid or a derivative thereof, preferably in a weight ratio of pinolenic acid to C20:4 PUFA of from 10:1 to 1:10, more preferably from 3:1 to 1:3, such as from 5:2 to 2:5 or from 3:1 to 1:1. These compositions preferably comprise from 0.5 to 20 wt %, more preferably from 1 to 15 wt %, such as from 2 to 10 wt % of arachidonic acid and from 0.5 to 40 wt %, more preferably from 1 to 30 wt %, such as from 5 to 20 wt % or 8 to 17 wt % of pinolenic acid. The compositions optionally further comprise from 20 to 60 wt %, more preferably from 25 to 55 wt %, such as from 30 to 50 wt % or 35 to 45 wt % linoleic acid. Another optional component is from 5 to 45 wt %, more preferably from 10 to 40 wt %, such, as from 15 to 35 wt % or 18 to 30 wt % of oleic acid. Additionally, the compositions preferably comprise from 5 to 15 wt % of saturated fatty acids and/or from 0.05 to 1 wt % trans fatty acids. Again, the fatty acids may be present in the compositions of the present inventions either as free acids or glycerides (e.g., mono-, di- or tri-glycerides). Similarly, weight percentages are calculated as free fatty acids based on the total fatty acid content of the composition and the balance is provided by the remaining fatty acids. In these compositions, the pinolenic acid can independently be present as a free fatty acid or as a derivative thereof, or as a mixture thereof. Derivatives of pinolenic acid are non-toxic and edible and preferably include, for example, salts and esters. Suitable salts include salts with food grade cations such as sodium salts. Suitable esters include alkyl esters having from one to six carbon atoms. Preferred esters are mono-, di- and tri-glycerides and mixtures thereof, with triglycerides being particularly preferred. The C20:4 PUFA or derivative thereof is optionally mixed with pinolenic acid or a derivative thereof before being used to prepare a composition according to the present invention.

The composition of the invention is preferably free of or substantially free of metal ions having an atomic number greater than 20, such as added metal ions. For example, the compositions preferably contain less than 0.0003 weight %, preferably less than 0.0002 weight %, such as less than 0.0001 weight % of these metal ions. The compositions may be free of chromium ions e.g., chromium complexes such as chromium picolinate or chromium nicotinate, chromic tripicolinate, chromic polynicotinate, chromium chloride, chromium histidinate and chromium yeasts. The composition is also preferably free of or substantially free of zinc ions, i.e., contains less than 1 mg, preferably less than 0.5 mg, such as less than 0.1 mg of zinc ions. For example, the composition preferably comprises less than 0.1 weight %, such as less than 0.05 weight % of zinc ions.

The C20:4 PUFA or derivative, optionally in combination with the further fatty acids or fatty acid derivatives, are preferably the only (i.e., sole) pharmacologically active agents in the composition of the present invention. For example, the composition is preferably free of or substantially free of, i.e., containing less than 1 weight %, such as less than 0.5 weight % or less than 0.3 weight % or less than 0.1 weight % of a lipase inhibitor, such as orlistat (tetrahydrolipstatin), panclicins and 2-oxy-4H-3,1-benzoxazin-4-ones (e.g., 2-decyloxy-6-methyl-4H-3,1-benzooxazin-4-one, 6-methyl-2-tetradecyloxy-4H-3,1-benzoxazin-4-one and 2-hexadecyloxy-6-methyl-4H-3,1-benzoxazin-4-one) and/or a diabetes medicine such as nateglinide, metformin or a 4-hydroxy-isoleucine source. For example, the composition of the invention preferably comprises less than 10 mg, such as less than 6 mg or less than 1 mg of a lipase inhibitor.

The compositions of the invention are preferably free or substantially free of vitamin K (for example, vitamin K is present in the compositions in an amount of less than 1000 μg/100 g (i.e., less than 0.001 weight %), such as from 0.1 to 900 μg/100 g or from 10 to 800 μg/100 g or from 30 to 600 μg/100 g).

The compositions of the invention are preferably free of or substantially free of free amino acids and/or salts thereof i.e., the compositions typically comprise less than 1% by weight, more preferably less than 0.1% by weight of free amino acids and/or salts thereof.

Most preferably, the compositions of the invention are free or substantially free of: added metal ions having an atomic number greater than 20; and lipase inhibitors; and anti-diabetic compounds; and vitamin K; and free amino acids and their salts.

A suitable source of pinolenic acid, which can be admixed with the C20:4 PUFA or derivative used in the compositions of the present invention, is pine nut oil or a concentrate thereof. For example, glycerides of pinolenic acid can be obtained from pine nut oil or concentrates thereof. Preferably, an oil or concentrate with a content of pinolenic acid or a derivative thereof of more than 10 wt % is used.

The skilled person will appreciate that the percentage of C20:4 PUFA or a derivative thereof in a composition of the invention will depend on the nature of the composition. For example, the C20:4 PUFA or derivative thereof is likely to represent a higher percentage of the total weight of a pharmaceutical composition or a food supplement than a food composition.

When the composition of the invention is in the form of a food supplement or a pharmaceutical product, the C20:4 PUFA or derivative thereof or a mixture containing one of these compounds may be in encapsulated form in a food grade encapsulating material. Suitable encapsulating materials are well known in the art and include, for example, gelatin and glycerol.

Preferably, compositions of the invention are free of (or substantially free of, i.e., containing less than 0.1 mg of) uridine esters, and/or nucleosides and/or nucleotides selected from the group consisting of uridine, deoxyuridine, uridine monophosphate, deoxyuridine monophosphate and/or pharmaceutically acceptable salts thereof.

The compositions of the invention may comprise one or more other fatty acids (i.e., straight chain saturated or unsaturated carboxylic acids having from 12 to 24 carbon atoms). Examples of other fatty acids suitable for use in the present invention include linoleic acid, oleic acid, taxoleic, juniperonic, sciadonic, saturated fatty acids, conjugated linoleic acid (optionally as an enriched isomer mixture) and EPA (eicosapentaenoic), DHA (docosahexaenoic) (optionally as an enriched isomer mixture of EPA or DHA) and conjugated trienoic acids such as GLA and ALA. Enrichment involves the alteration of the isomer mixture normally present (for example in a natural product), such as an alteration in the relative amounts of different geometrical isomers. In these compositions, the other fatty acid or each of the other fatty acids can independently be present as a free fatty acid or as a derivative thereof (including a mono-, di- or triglyceride and salts), or as a mixture thereof.

The C20:4 PUFA or derivative thereof is optionally blended with these additional fatty acids or glycerides before being used to prepare a composition according to the present invention. When the compositions of the invention contain one or more fatty acids and/or glycerides in addition to the C20:4 PUFA or derivative thereof, the additional fatty acid(s) and/or glycerides are preferably selected from liquid oils, such as soybean oil, sunflower oil, rape seed oil and cotton seed oil pomegranate seed oil; cocoa butter and cocoa butter equivalents; palm oil and fractions thereof; enzymically made fats; fish oils and fractions thereof; conjugated linoleic acid and enriched isomer mixtures; gamma linolenic acid and enriched mixtures thereof; hardened liquid oils; and mixtures thereof.

Blends containing one or more additional fatty acids or glycerides, that are typically used in food compositions of the invention, preferably display solid fat contents measured by NMR-pulse on non stabilised fats of;

N20=1-80, preferably 5-45
N35 less than 20, preferably less than 10, most preferably less than 5.

These values were obtained by melting the fat at 80° C., cooling to 0° C. and holding the fat at 0° C. for 30 minutes, whereupon the fat was heated to the measurement temperature N and held at that temperature for 30 minutes before measuring the N value.

Blends of fatty acids that are used to produce the compositions of the invention may comprise from 1.5 to 60 wt %, more preferably from 28 to 60 wt % of a C20:4 PUFA or derivative thereof, from 10 to 60 wt % of linoleic acid and from 5 to 52 wt % of oleic acid, for example 25 to 85 wt % (linoleic plus oleic acid), from 0 to 70 wt %, for example 25 to 65 wt % (trans plus saturated fatty acid). The blends optionally further comprise from 1.5 to 60 wt %, more preferably from 14 to 30 wt % pinolenic acid. The trans fatty acid content is preferably less than 10 wt %. An example of a suitable blend is one in which the trans plus saturated fatty acid content is less than 10 wt %.

Alternatively, the compositions of the invention may be free or substantially free of fatty acids other than the C20:4 PUFA (i.e., may contain less than 1% by weight, more preferably less than 0.1% by weight, such as less than 0.01% by weight of other C12 to C24 fatty acids).

The compositions of the invention may comprise calcium and/or magnesium sources. Additionally or alternatively, the compositions may comprise protein (including protein hydrolysates). The combination of a C20:4 PUFA and calcium and/or magnesium and/or protein in the compositions of the present invention may increase the release of CCK from the intestinal cells.

In another embodiment, the compositions of the invention may be free or substantially free of calcium ions and/or protein (i.e., each of these components is present in the compositions in an amount of less than 1% by weight, more preferably less than 0.1% by weight, such as less than 0.01% by weight).

The compositions of the present invention can be used to help manage body weight, for example to help maintain body weight at a substantially constant level or to help reduce body weight. In other words, use of the compositions can assist in slimming the body, for example by helping to induce fat loss.

The use of the compositions of the invention can help to reduce calorie intake. This can help maintain body weight at a substantially constant level and/or can help reduce body weight and/or help slimming. Reduction in body weight can increase energy levels.

The use of the compositions of the invention can reduce the feeling of hunger and/or increase satiety and/or reduce the desire for high calorie foods, for example by allowing less room in the stomach for high calorie foods. In particular, the use of the compositions of the invention can enhance and/or extend satiety or fullness prior to, during and/or after a meal.

The use of the compositions of the invention can reduce the feeling of hunger during dieting and therefore increase the success rate of a diet.

Consumption of the composition of the invention can stimulate the release of any one of the following: CCK, IgA, PYY (peptide tyrosine-tyrosine) or gLP (glucagon-like peptide), or any combination thereof.

The use of the compositions of the invention can help to reduce appetite, for example by increasing satiation and a feeling of satiety and/or fullness.

The present invention also provides a method of treating or preventing obesity. In this method an effective amount of a composition as described above is orally administered to a mammal, for example a human. The mammal is preferably in need of the treatment (e.g., is obese, overweight or has difficulty in avoiding weight gain). The present invention also provides a method of weight management. In this method an effective amount of a composition as described above is orally administered to a mammal, for example a human. Said administration need not be carried out by a physician or under medical supervision and can simply involve the mammal ingesting the composition e.g., in the form of a foodstuff or food supplement. Preferably, the compositions of the invention are administered (or taken) 2 hours to 3 minutes, more preferably 1 hour to 15 minutes and even more preferably 35 to 25 minutes before eating a meal.

The invention is applicable to mammals, preferably humans. Other mammals that may benefit from the compositions of the invention include pets (for example, dogs, cats, horses, rabbits, hamsters and guinea pigs) and farm animals (for example, cattle, sheep and pigs). Dogs and cats are particularly preferred.

When producing a food product, the C20:4 PUFA or derivative thereof can first be blended with structuring components for use in food products. However, this is not essential. These blends can, for example, be applied beneficially in food products as healthy fat compositions.

The compositions of the invention can be food products. Food products in which a C20:4 PUFA or derivative thereof or blends containing these compounds can be used include, but are not limited to: margarines; low fat spreads; very low fat spreads; bicontinuous spreads; water continuous spreads; confectionery products, such as chocolates, coatings or fillings; ice creams; ice cream coatings; ice cream inclusions; dressings; mayonnaises; sauces; bakery fats; shortenings; cheese; meal replacement products; health bars; muesli bars; drinks; dairy products; low carbohydrate products; low calorie products; soups; cereals; and milk shakes.

The addition of a C20:4 PUFA or a derivative thereof or blends containing at least one of the compounds to food products can have a positive effect on the texture, hardness, appearance, rheology, oral melt, flavour impact, spreadability, microstructure (crystal size, droplet size), aeration properties or ease of processing of these food products. The use of a glyceride of the C20:4 PUFA used in the compositions of the present invention is particularly advantageous in this respect.

Other examples of compositions are pharmaceutical compositions, such as in the form of tablets, pills, capsules, caplets, multiparticulates including: granules, beads, pellets and micro-encapsulated particles; powders, elixirs, syrups, suspensions and solutions. Pharmaceutical compositions will comprise a pharmaceutically acceptable diluent or carrier. Pharmaceutical compositions are preferably adapted for administration parenterally (e.g., orally). Orally administrable compositions may be in solid or liquid form and may take the form of tablets, powders, suspensions and syrups. Optionally, the compositions comprise one or more flavouring and/or colouring agents. Pharmaceutically acceptable carriers suitable for use in such compositions are well known in the art of pharmacy. The compositions of the invention may contain 0.1-99% by weight of a C20:4 PUFA or derivative. The compositions of the invention are generally prepared in unit dosage form. Preferably, the unit dosage of the C20:4 PUFA or derivative thereof is from 1 mg to 1000 mg (more preferably from 100 mg to 750 mg). The excipients used in the preparation of these compositions are the excipients known in the art.

Further examples of product forms for the composition are food supplements (which term includes nutritional products), such as in the form of a soft gel or a hard capsule comprising an encapsulating material selected from the group consisting of gelatin, glycerol, starch, modified starch, starch derivatives such as glucose, sucrose, lactose and fructose. The encapsulating material may optionally contain cross-linking or polymerizing agents, stabilizers, antioxidants, light absorbing agents for protecting light-sensitive fills, preservatives and the like. Preferably, the unit dosage of the C20:4 PUFA or derivative thereof in the food supplements is from 1 mg to 1000 mg (more preferably from 25 mg to 200 mg, such as from 50 mg to 150 mg, most preferably from 75 mg to 125 mg).

The compositions of the invention may contain other additives that are well known in the art of food and pharmaceutical products including, but not limited to, flavouring ingredients, colouring agents, sweeteners and emulsifiers.

The following non-limiting examples illustrate the invention and do not limit its scope in any way. In the examples and throughout this specification, all percentages, parts and ratios are by weight unless indicated otherwise.

### EXAMPLES

### Example 1

The relative CCK release by a number of different compounds was determined in an in-vitro trial to measure the effect of various free fatty acids on CCK release from intestinal cells. The study showed the effect of arachidonic acid as a satiety ingredient.

### Cell Culture

The enteroendocrine STC1-1 cell line was cultivated at 37° C. in a 5% CO₂, 95% air atmosphere in standard culture medium (DMEM). Cells were routinely passaged upon reaching 70-80% confluency by washing the cell layer with PBS and incubating with a solution of trypsin-EDTA. Plating density of 2×10⁶ cells by 75 cm² was used for routine subculture.

### Experimental Protocol

STC1 cells were seeded into 6-well culture plates and incubated with control culture medium, with or without the tested agents, for 1 hour. All agents were tested at a 100 μM concentration in the free fatty acid form. Since fatty acids were diluted into ethanol the effect of ethanol was tested and indicated the baseline CCK levels. Capric acid was used as a negative control fatty acid, because it is already known that capric acid does not induce CCK. Effects of bombesin was used as positive control. At the end of incubation the supernatant was collected, centrifuged and immediately frozen at −20° C. for RIA.

Cell viability was checked by microscopic analysis using an MTT cytotoxicity assay, and in addition LDH release was measured.

### RIA Analysis

CCK release was measured using a standard CCK RIA and effects of fatty acids were measured in 6-fold.

Table 1 shows that arachidonic acid surprisingly produced a higher level of CCK release than other related fatty acids.

TABLE 1
Effects of fatty acids on CCK release

| | | 100 | |
|---|---|---|---|
| | Fatty acid | microM | SEM* |
| 1 | Pinolenic acid (16%) | 188 | 17 |
| 2 | Pinolenic acid (27%) | 210 | 23 |
| 6 | Oleic acid (18:1) | 90 | 7 |
| 7 | Linoleic acid (18:2) | 68 | 11 |
| 8 | Taxoleic acid (18:2) | 82 | 9 |
| 9 | Eicosapentanoic acid (20:5) | 164 | 14 |
| 10 | Sciadonic acid (20:3) | 79 | 3 |
| 11 | Juniperonic acid (20:4) | 97 | 8 |
| 12 | Gamma-linolenic acid (18:3) | 140 | 6 |
| 13 | Alpha-linolenic acid (18:3) | 177 | 8 |
| 14 | Alpha-eleostearic acid (18:3) | 212 | 5 |
| 15 | Gamma-eleostearic acid (18:3) | 126 | 5 |
| 16 | Punicic acid (18:3) | 320 | 14 |
| 17 | Stearidonic acid (18:4) | 69 | 10 |
| 18 | Arachidonic acid (20:4) | 392 | 25 |
| 19 | Commercial product, based on | 91 | 3 |
| | oats) | | |
| 20 | CLA | 87 | 6 |
| 21 | CLA 9, 11 isomer | 135 | 2 |
| 22 | CLA 10, 12 isomer | 133 | 6 |
| 23 | CLA/pinolenic acid | 112 | 7 |
| 24 | Lauric acid | 118 | 4 |
| 25 | Capric acid | 43 | 6 |
| 26 | Ethanol | 53 | 17 |

*Standard Error Mean

### Example 2

The following is an example of a filled gelatin capsule according to the invention. A composition comprising arachidonic acid is encapsulated into a gelatin capsule according to methods well-known in the art. The resulting encapsulated product contains 500 mg of arachidonic acid and one capsule can be taken up to four times daily by an adult human.

### Example 3

The following is an example of a food supplement comprising 225 mg pinolenic acid in admixture with 112.5 mg arachidonic acid. A composition comprising 1.5 g pine nut oil and 240 mg arachidonic oil (commercially available fungal oil) is encapsulated into a gelatin capsule according to methods well-known in the art. The composition has the following fatty acid composition (by weight based on total fatty acid content):

| | |
|---|---|
| Oleic acid | 23% |
| Linoleic acid | 41% |
| Pinolenic acid | 13% |
| Arachidonic acid | 6.5% |
| Balance | 16.5% |
| Total monounsaturated fatty acids | 24% |
| Total polyunsaturated fatty acids | 65% |
| Total saturated fatty acids | 10% |
| Total trans fatty acid | 0.2% |

### Example 4

The following is an example of a food supplement according to the invention. A vegetable fungal oil containing 47% by weight arachidonic acid is encapsulated into a gelatin capsule according to methods well-known in the art. The oil has the following fatty acid compositions (by weight based on total fatty acid content):

| | |
|---|---|
| Arachidonic acid | 47% |
| Palmitic (C16:0) | 6.3% |
| Stearic (C18:0) | 4.8% |
| Docosanoic (C22:0) | 3.0% |
| Tetracosenoic (24:0) | 9.4% |
| Oleic (C18:1) | 9.4% |
| Linoleic (C18:2) | 4.2% |
| Homogamma linolenic (C20:3) | 3.7% |
| Balance | 12.2% |

1. (canceled)

2. A method according to claim 20, wherein the composition is free or substantially free of metal ions having an atomic number greater than 20.

3. A method according to claim 2, wherein the composition is in the form of a food supplement, a pharmaceutical composition or a food composition.

4. A method according to claim 3, wherein the composition comprises from 0.5 to 40% by weight of the total fatty acid content of the composition of oleic acid in the form of a free fatty acid or glyceride.

5. A method according to claim 4, wherein the composition comprises from 0.5 to 20% by weight of the total fatty acid content of the composition of linoleic acid in the form of a free fatty acid or glyceride.

6. A method according to claim 20, wherein the composition further comprises pinolenic acid or a derivative thereof.

7. A method according to claim 6, wherein the composition comprises from 5 to 45% by weight of the total fatty acid content of the composition of oleic acid in the form of a free fatty acid or glyceride.

8. A method according to claim 6, wherein the composition comprises from 20 to 60% by weight of the total fatty acid content of the composition of linoleic acid in the form of a free fatty acid or glyceride.

9. A method according to claim 6, wherein the pinolenic acid is present as a free fatty acid, a mono-, di- or triglyceride, or a mixture thereof.

10. A method according to claim 20, wherein the arachidonic acid is derived from algae.

11. A method according to claim 20, wherein the composition additionally comprises a fatty acid or derivative thereof selected from the group consisting of linoleic acid, oleic acid, conjugated linoleic acid, enriched isomer mixtures of conjugated linoleic acid, EPA, DHA, conjugated trienoic acids such as GLA or ALA, or a mixture thereof.

12. A method according to claim 11, wherein the fatty acid or derivative thereof is present as a free fatty acid, a mono-, di- or triglyceride, or a mixture thereof.

13. A method according to claim 20, wherein the composition further comprises at least one glyceride formed from linoleic acid, oleic acid, trans acids and saturated fatty acids and the composition is a food composition.

14. A method according to claim 13, wherein the glyceride is selected from liquid oils selected from soybean oil, sunflower oil, rape seed oil and cotton seed oil; cocoa butter and cocoa butter equivalents; palm oil and fractions thereof; enzymically made fats; fish oils and fractions thereof; conjugated linoleic acid and enriched isomer mixtures thereof; gamma linoleic acid and enriched mixtures thereof; hardened liquid oils; and mixtures thereof.

15. A method according to claim 20, wherein the composition is a food product selected from the group consisting of: margarines; low fat spreads; very low fat spreads; bicontinuous spreads; water continuous spreads; confectionery products, such as chocolates, coatings or fillings; ice creams; ice cream coatings; ice cream inclusions; dressings; mayonnaises; sauces; bakery fats; shortenings or cheese; meal replacement products; health bars; muesli bars; drinks; dairy products; low carbohydrate products; low calorie products; soups; cereals and milk shakes.

16. A method according to claim 20, wherein the composition is a pharmaceutical composition in a form selected from the group consisting of tablets, pills, capsules, caplets, multiparticulates including: granules, beads, pellets and micro-encapsulated particles; powders, elixirs, syrups, suspensions and solutions.

17. A method according to claim 20, wherein the composition is a food supplement in the form of a soft gel or a hard capsule comprising an encapsulating material selected from the group consisting of gelatin, starch, modified starch, and starch derivatives.

18. A method according to claim 20, wherein the composition is for weight management by helping to maintain body weight and/or to reduce body weight and/or assists in slimming the body and/or reduces calorie intake and/or allows less room for high calorie foods.

19. A method according to claim 20 wherein the composition is for reducing the feeling of hunger and/or increasing satiety and/or increasing the production of cholecystokinin (CCK) and/or IgA.

20. Method of treating or preventing obesity or for weight management comprising orally administering an effective amount of an orally administrable composition comprising arachidonic acid in the form of an alkyl ester having from 1 to 6 carbon atoms, a mono-, di- or triglyceride or a mixture thereof to a mammal.

21. Method of weight management according to claim 20 comprising orally administering an effective amount of a composition an orally administrable composition comprising arachidonic acid in the form of an alkyl ester having from 1 to 6 carbon atoms, a mono-, di- or triglyceride or a mixture thereof to a mammal.

22. Method of stimulating the production of cholecystokinin (CCK) comprising orally administering an effective amount of a composition an orally administrable composition comprising arachidonic acid in the form of an alkyl ester having from 1 to 6 carbon atoms, a mono-, di- or triglyceride or a mixture thereof to a mammal.

23. Method as claimed in claim 20, 21 or 22, wherein the mammal is a human.

24. Composition for oral administration comprising: from 0.5 to 20 wt % of arachidonic acid or a derivative thereof; and from 0.5 to 40 wt % of pinolenic acid or a derivative thereof.

25. Composition according to claim 24 comprising from 1 to 15 wt % of arachidonic acid or derivative thereof, and from 1 to 30 wt % of pinolenic acid or derivative thereof.
